(19) United States
(12) Patent Application Publication
(10) Pub. No.: US 2017/0252023 A1
(43) Pub. Date: Sep. 7, 2017

(54) SURGICAL INSTRUMENT FOR COLORECTAL POLYP REMOVAL

(71) Applicant: GLENN MARK MCALPIN, NEWNAN, GA (US)

(72) Inventor: GLENN MARK MCALPIN, NEWNAN, GA (US)

(21) Appl. No.: 15/451,383

(22) Filed: Mar. 6, 2017

Related U.S. Application Data
(60) Provisional application No. 62/303,485, filed on Mar. 4, 2016.

(51) Int. Cl. A61B 10/04; A61B 1/31; A61B 1/018; A61B 10/02; A61B 10/06; A61B 18/14

(52) U.S. Cl. CPC A61B 10/04; A61B 10/06; A61B 18/1492; A61B 1/018; A61B 10/0266; A61B 1/31; A61B 2018/00595

(57) ABSTRACT

Disclosed are various embodiments of a surgical instrument for colorectal polyp removal. In various embodiment, the surgical instrument has an elongate flexible tubular sheath having a proximal end and a distal end, a snare having a loop and controlled by a pair of wires, and a biopsy forceps having a pair of jaws sized to fit within the loop of the snare. Both the biopsy forceps and the snare are routed through the elongate flexible tubular sheath so that the loop of the snare and the pair of jaws exit the distal end of the elongate flexible tubular sheath.

### CROSS-REFERENCE TO RELATED APPLICATIONS

This application claims priority to, co-pending U.S. Provisional Application entitled “SURGICAL INSTRUMENT FOR COLORECTAL POLYP REMOVAL,” filed on Mar. 4, 2016, and assigned application No. 62/303,485, which is incorporated herein by reference in its entirety.

### BACKGROUND

A colorectal polyp is a fleshy growth occurring on the lining of the colon or rectum. Untreated colorectal polyps can develop into colorectal cancer. Polyps may be classified according to their behavior or etiology. Some may be benign, while others may be malignant. Polyps can be removed during a colonoscopy or sigmoidoscopy.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is an example of a perspective view of a surgical instrument according to an embodiment of the present disclosure.

FIG. 2 is an example of a top view of the surgical instrument of FIG. 1 according to an embodiment of the present disclosure.

FIG. 3 is an example of an end view of the surgical instrument of FIG. 1 according to an embodiment of the present disclosure.

FIG. 5 is a flowchart depicting an example method of use for the surgical instrument of FIG. 1 according to an embodiment of the present disclosure.

### DETAILED DESCRIPTION

The present disclosure relates to a surgical instrument that improves acquisition and removal of polyps, especially pedunculated polyps and some sessile polyps. Pedunculated polyps are those having a tubular appearance and attached to the intestinal wall by a stalk, while sessile polyps are those that are flat appearing and grow directly from the wall. Typically, polyps can be removed using a snare tool comprising a wire loop that cuts the stalk of the polyp and cauterizes it to prevent bleeding. However, using a snare tool alone often requires multiple attempts to remove a polyp.

Specifically, in one embodiment, a surgical instrument is disclosed that is a combination of a snare tool and biopsy (BX) forceps. The surgical instrument facilitates retrieval of an entire polyp without the multiple attempts usually required in using, for example, a snare tool alone. Removal of polyps with a snare tool may require multiple attempts due to the difficulty in placing the snare around the polyp completely. By placing BX forceps within the snare loop, this problem will be resolved. The BX forceps will retrieve and stabilize the entire polyp for snare placement and removal.

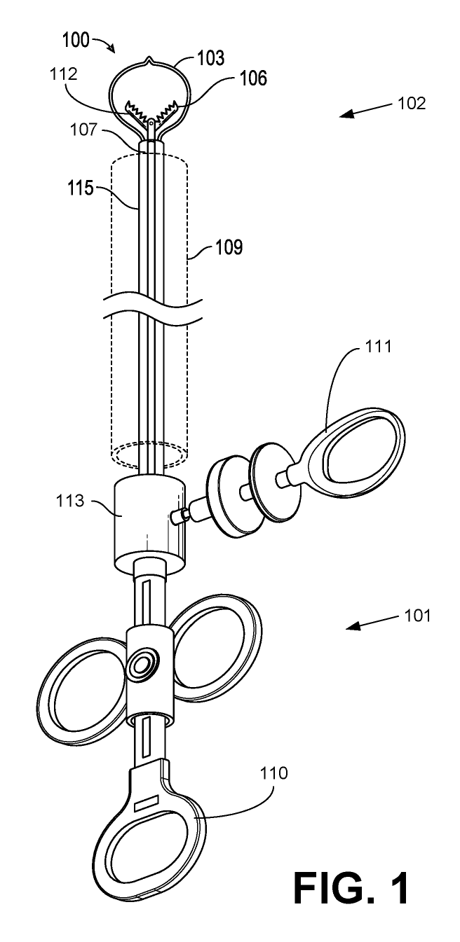

Referring now to FIG. 1, shown is an example of a perspective view of a surgical instrument 100 according to an embodiment of the present disclosure. The surgical instrument 100 has a proximal end 101 and a distal end 102. The surgical instrument 100 comprises a combination of a snare 103 and a BX forceps 106, within a shaft 107, which is configured to be placed within a plastic core 109 or elongate tubular member, such as a catheter sheath for endoscopy. The BX forceps 106 may be placed within the snare 103 loop. Otherwise, the BX forceps 106 may exit the core 109 alongside the snare 103.

The snare 103 and BX forceps 106 have separate coils and are able to move independently. Control handle 110 is used to control the snare 103, while control handle 111 is used to control the BX forceps 106.

The BX forceps 106 when activated is mobile (in and out) to grab the polyp using a pair of jaws 112. At 112, the wires of the snare 103 fuse to form a rigid rod for better movement. The wires of the snare 103 are made hot via a source of electrocautery current to perform the cauterization function of the polyp during removal. At 115, there is some stiffness to the wires to control the snare 103. It is noted that the snare 103 comprises two separate wires instead of one.

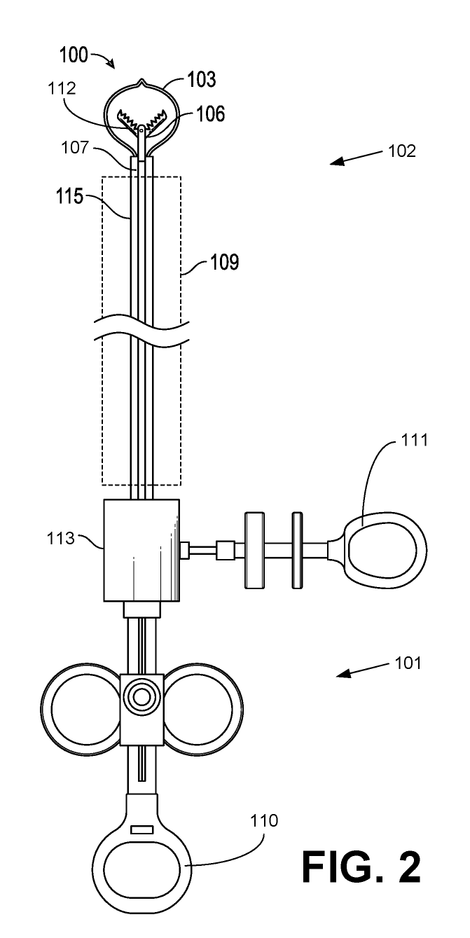

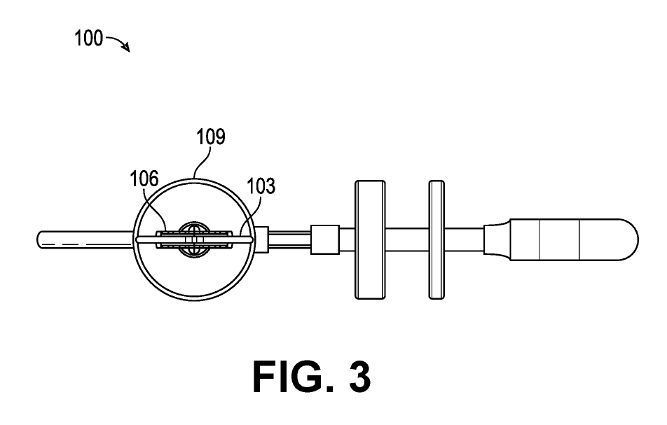

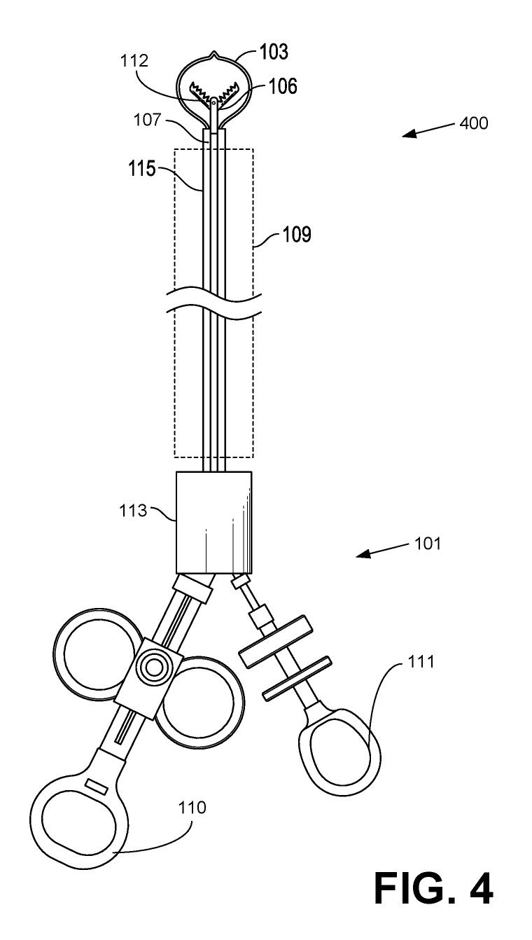

FIG. 2 provides a top view of the surgical instrument 100 according to an embodiment of the present disclosure. FIG. 3 provides an end view of the plastic core 109, the snare 103, and the BX forceps 106.

FIG. 4 provides a top view of a surgical instrument 400 constituting a variation on the surgical instrument 100. While in FIG. 2, the control handle 111 exits the shaft 107 at a right angle relative to the control handle 110, in FIG. 4, the control handles 110 and 111 are loosely coupled to the shaft 107.

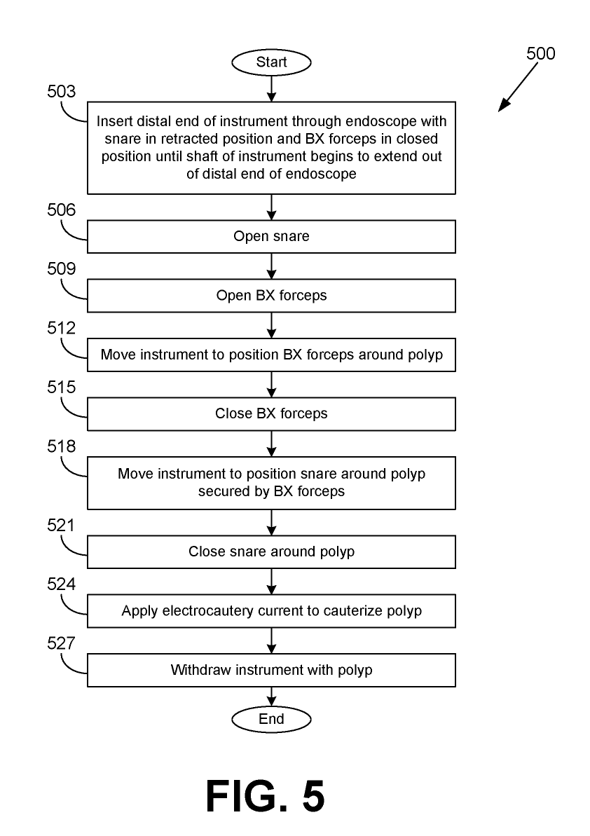

FIG. 5 is a flowchart describing one example method of use 500 for the surgical instrument 100. At 503, the distal end 102 of the instrument 100 is inserted through an endoscope within the snare 103 in a retracted position and the BX forceps 106 in a closed position until the shaft 107 of the instrument 100 begins to extend out of the distal end of the endoscope. At 506, the snare 103 is opened using the control handle 110. At 509, the BX forceps 106 are opened using the control handle 111. At 512, the instrument 100 is moved to position the BX forceps 106 around a polyp. For example, the shaft 107 can be advanced and retracted into and out of the endoscope, while torque to the instrument 100 can be applied. At 515, the BX forceps 106 are closed using the control handle 111.

At 518, the instrument 100 is moved in order to position the snare 103 around the polyp, which is secured by the BX forceps 106. For example, the shaft 107 can be advanced and retracted into and out of the endoscope, while torque to the instrument 100 can be applied. At 521, the snare 103 is closed around the polyp using the control handle 110. At 524, electrocautery current is applied to desiccate, sever, and cauterize the polyp. At 527, the instrument 100 is withdrawn with the polyp being removed.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A surgical instrument for polypectomy, comprising:
an elongate flexible tubular sheath having a proximal end and a distal end;
a snare having a loop and controlled by a pair of wires;
a biopsy forceps having a pair of jaws sized to fit within the loop of the snare; and
wherein both the biopsy forceps and the snare are routed through the elongate flexible tubular sheath so that the loop of the snare and the pair of jaws exit the distal end of the elongate flexible tubular sheath.

2. The surgical instrument of claim 1, further comprising a source of electrocautery current coupled to the pair of wires of the snare, wherein when activated the electrocautery current causes the loop of the snare to heat.

3. The surgical instrument of claim 1, further comprising a first control handle configured to control opening and closing of the loop of the snare, and a second control handle configured to control opening and closing of the pair of jaws of the biopsy forceps.

4. The surgical instrument of claim 3, wherein the second control handle is mounted at a right angle relative to the first control handle at a rigid member of the surgical instrument.

5. The surgical instrument of claim 3, wherein the first control handle and the second control handle are loosely coupled at an opening at the proximal end of the elongate flexible tubular sheath.

6. A method for polypectomy, comprising:
inserting a distal end of a surgical instrument through an endoscope, the surgical instrument comprising:
an elongate flexible tubular sheath having a proximal end and a distal end;
a snare having a loop and controlled by a pair of wires;
a biopsy forceps having a pair of jaws sized to fit within the loop of the snare; and
wherein both the biopsy forceps and the snare are routed through the elongate flexible tubular sheath so that the loop of the snare and the pair of jaws exit the distal end of the elongate flexible tubular sheath, and the snare is in a retracted position and the biopsy forceps is in a closed position;
opening the snare;
opening the biopsy forceps;
positioning the pair of jaws of the biopsy forceps around a polyp;
closing the pair of jaws of the biopsy forceps on the polyp, thereby securing the polyp;
positioning the loop of the snare around the polyp that has been secured by the biopsy forceps; and
closing the loop of the snare around the polyp.

7. The method of claim 6, further comprising applying an electrocautery current to the snare to cauterize the polyp.

8. The method of claim 6, further comprising withdrawing the surgical instrument through the endoscope.
